(19) United States
(12) Patent Application Publication
(10) Pub. No.: US 2010/0311109 A1
(43) Pub. Date: Dec. 9, 2010

(54) NON-CONTACT METHOD FOR QUANTIFYING CHANGES IN THE DYNAMICS OF MICROBIAL POPULATIONS

(76) Inventors: Ahmad A. Salaimeh, Lexington, KY (US); Martin E. Evans, Richmond, KY (US); Jefferey J. Campion, Lexington, KY (US); Belal M. Gharaibeh, Lexington, KY (US); Kozo Saito, Lexington, KY (US)

Correspondence Address: KING & SCHICKLI, PLLC, 247 NORTH BROADWAY, LEXINGTON, KY, 40507, US

(21) Appl. No.: 12/477,384

(22) Filed: Jun. 3, 2009

(51) Int. Cl. C12Q 1/06; C12Q 1/02

(52) U.S. Cl. 435/39; 435/29

(57) ABSTRACT

A method for quantifying an amount of a viable microorganism includes subjecting a fluid sample suspected of containing a viable microorganism to a temperature change, and correlating the temperature history of the fluid sample to the amount of the viable microorganism contained in the fluid sample. The method may include the steps of bringing, the fluid sample to a first temperature, and transferring the fluid sample to a second temperature that is different than the first temperature. After the step of transferring, next is the step of measuring a temperature change in the fluid sample over a predetermined period of time. The temperature change may then be correlated to the amount of the viable microorganism contained in the fluid sample. The method finds use in a variety of applications, including evaluation of compositions or compounds potentially having microbicidal, microbiostatic, or growth enhancing properties.

### TECHNICAL FIELD

The present invention relates to methods for quantifying changes in viable microbial populations. In particular, the invention relates to real-time methods for quantifying such alterations in microbial populations, and for rapid quantification of viable microorganism in situ. The invention finds use in a variety of applications where living organisms, suspended in a liquid medium, are quantified, including evaluation of antimicrobial agents and/or microbial growth enhancers.

### BACKGROUND OF THE INVENTION

Quantification of microorganisms is a critical element in a number of microbiological, pharmaceutical, ecological, and industrial settings. Specific examples include evaluation of antimicrobial susceptibility and/or antimicrobial efficacy, development of novel antimicrobials, wastewater treatment, water quality assessment, and food safety applications such as detection of contaminated food products, pasteurization quality control, and quality control in fermentation processes. Reliable methods for quantification of microorganisms, particularly methods capable of concurrently distinguishing between living and dead microorganisms are essential tools in such endeavors.

Such microorganism quantification is most commonly done by colony count methods requiring prolonged incubation times for appearance of colonies on growth media. Indeed, colony count methods are at present the most definitive and reliable method available for bacterial detection and quantification in culture. Alternatively, it is known also to quantify microorganisms by microscopic imaging. These processes are labor and resource intensive, and conventional microscopic imaging processes suffer from the further disadvantage of failing to differentiate between living and dead cells absent introduction of a chemically enhanced substance. Conventional spectrophotometric methods similarly fail to differentiate between viable and non-viable cells, typically applicable to aqueous media, precluding their use in the food industry, and are also limited to a 2-3 log range of cell concentrations in terms of their ability to quantitate.

Impedance and pH methods for quantification of microorganisms are suited thr their intended purpose, but suffer from limitations in range of detection due to material chemical property limitations to allowable pH and changes in pH. Other methods are known or in various stages of development, such as calorimetric methods, enzyme labeling, gene sensors, and flow injection, but require chemical treatment or complete destruction of samples, preventing dynamic change measurements.

Indeed, each of the conventional methods discussed above suffer from a common deficiency, that is, inability to quantify viable microorganisms over a wide dynamic range in real-time. Thus, there remains a need in the art for novel methods for quantification of microorganisms, in particular methods allowing distinguishing viable from non-viable cells.

### SUMMARY OF THE INVENTION

To solve the aforementioned and other problems, there is provided a method for quantifying an amount of a viable microorganism, comprising subjecting a fluid sample suspected of containing a viable microorganism to a temperature change and correlating the temperature change in the fluid sample to an amount of the viable organism in the fluid sample. In one embodiment, the method may be accomplished by the steps of bringing the fluid sample to a first temperature, and transferring the fluid sample to a second temperature that is different than the first temperature. Next is the step of measuring a temperature change in the fluid sample, and correlating that temperature change to the amount of the viable microorganism contained in the test fluid.

In another aspect, there is provided a method for determining the effect of a test substance on growth or viability of a microorganism, comprising suspending a predetermined amount of a viable microorganism in a fluid sample and adding a predetermined amount of the test substance to fluid sample. The fluid sample is then subjected to a temperature change, and the temperature change in the fluid sample is correlated to the amount of the viable microorganism contained in the fluid sample as described above to determine the effect of the test substance on the amount of the viable microorganism contained in the test fluid.

In one embodiment, the first temperature is maintained for a sufficient period of time to place the fluid sample in a state of thermal equilibrium. The temperature change may be measured by holding the fluid sample at the second temperature, optionally for a sufficient time to allow the fluid sample to reach thermal equilibrium. During the step of allowing the fluid sample to reach thermal equilibrium, the temperature change in the fluid sample is measured at spaced time intervals over a predetermined time period. That measuring step may be accomplished by acquiring a plurality of sequential thermal images of the fluid sample at the spaced time intervals, such as by infrared thermography.

The correlation step includes relating a plotted slope of the normalized temperature change (measured as described above) against the normalized predetermined time period to an amount of thermal energy released from the fluid sample. In one embodiment, the amount of thermal energy released from the fluid sample is correlated to the plotted slope of normalized temperature change against normalized predetermined time period according to the formula:

$${E}_{n} = \frac{Δ f ( T )}{Δ g ( t )}$$

where Δf(T) is change in normalized temperature and Δg(t) is change in normalized time.

These and other embodiments, aspects, advantages, and features of the present invention will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims. Various patent and non-patent citations are referenced herein. Unless otherwise indicated, any such citations are specifically incorporated by reference in their entirety into the present disclosure.

### BRIEF DESCRIPTION OF THE DRAWINGS

The accompanying drawings incorporated in and forming a part of the specification, illustrate several aspects of the present invention, and together with the description serve to explain the principles of the invention. In the drawings:

FIG. 1 shows a schematic representation of a system for accomplishing the present invention;

FIG. 2 shows thermal properties of Mueller-Hinton broth (MHB) in the presence and absence of Escherichia coli;

FIG. 3 shows a plot of viable bacterial count versus Energy Number (Eₙ);

FIG. 4 shows a time evolution of bacteria average temperature for increasing bacterial concentrations;

FIGS. 5a-b show growth curves for S. aureus measured by IRT (FIG. 5a) and by viable count method (FIG. 5b);

FIG. 6 shows thermal properties of growth media (MHB) with and without yeast, expressed as Eₙ;

FIGS. 7a-c present time-kill curves for E. coli exposed to varying amounts of ciprofloxacin (CIP); 7a) CIP time-kill curve for E. coli (CIP Minimum Inhibitory Concentration (MIC)=0.016 μg/mL) expressed as colony count results, 7b) CIF time-kill curve for E. coli (CIP MIC)=0.016 μg/mL) expressed as Eₙ obtained by infrared thermography (IRT) according to the present invention; 7c) relationship of viable counts and IRT results by regression analysis;

FIGS. 8a-b show cefazalin time-kill profiles for E. coli ATCC 25922, measured by IRT (FIG. 8a) and by viable count method (FIG. 8b); and

FIGS. 9a-b show cefazalin time-kill profiles for S. aureus ATCC 29213, measured by IRT (FIG. 9a) and by viable count method (FIG. 9b).

### DETAILED DESCRIPTION OF THE ILLUSTRATED EMBODIMENTS

In the following detailed description of the illustrated embodiments, reference is made to the accompanying drawings that form a part hereof, and in which is shown by way of illustration, specific embodiments in which the invention may be practiced. These embodiments are described in sufficient detail to enable those skilled in the art to practice the invention. Also it is to be understood that other embodiments may be utilized and that process, reagent, software, and/or other changes may be made without departing from the scope of the present invention.

A basic property of life is the generation of heat resulting from the biochemical reactions required for cellular function. For that reason, detection of heat generation, such as by calorimetry, has been considered as a tool to detect and quantify the number of microorganisms in biological samples. However, to date such methods necessitate complete destruction of the sample to measure energy content, precluding dynamic temperature change measurements. Likewise, infrared thermography (IRT) has been evaluated to detect heat production/dissipation in animals, plants, cells in culture, and cell-free systems (see U.S. Pat. No. 6,983,752, incorporated herein by reference). However, such methods do not contemplate monitoring changes in viable microorganism population dynamics overtime, and further provide no indication of suitability for quantification of such viable microorganism populations.

The present studies were designed to evaluate whether such generation of heat by microorganisms could provide a useful tool in non-contact quantification of microorganisms, especially viable microorganisms. The present investigators have found that the number of viable microorganisms in culture may be quantified by measuring energy transfer from a fluid containing microorganisms to the ambient surroundings. There is accordingly described herein a real-time, non-contact method for quantification of viable microorganisms by detecting such energy transfer by capture of sequential thermal images such as by infrared theithography. Use of the described method is contemplated for a variety of applications, including without limitation evaluation of potential microbiocidal and/or microhiostatic compositions or compounds, or alternatively for testing and evaluation of potential growth factors for microorganisms.

### Example 1

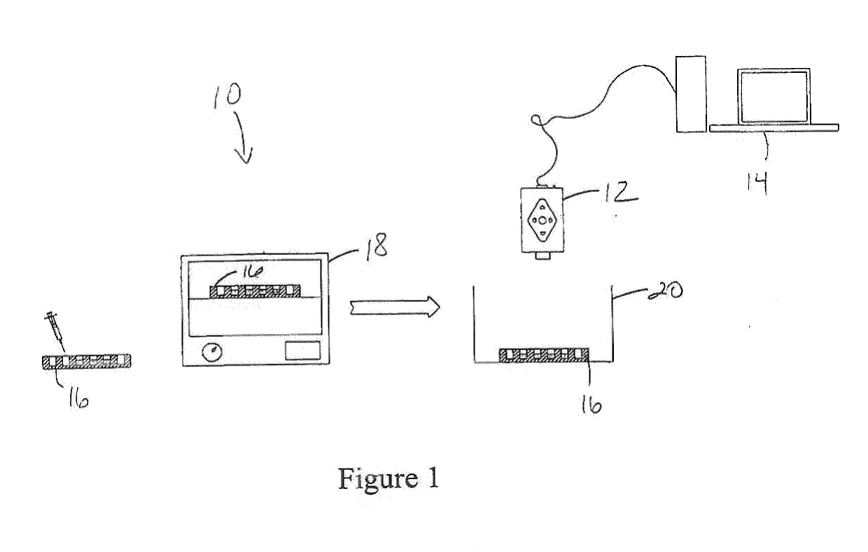

A system 10 for measuring energy transfer from bacterial cultures was developed, and is shown in schematic form in FIG. 1. The system utilizes an infrared detector 12 operatively connected to a data processor 14 for analyzing data obtained from the infrared detector 12 using a proprietary code. A sample holder 16 is provided, in the depicted example being a multi-well culture plate, such as for example a 24 or 96 well culture plate, into which fluids containing microorganisms and also control fluids may be co-cultured. The system 10 further includes an incubator 18 for holding one or more sample holders 16 at a first temperature, and a cooler 20 for holding one or more sample holders 16 at a second temperature that is less than the first temperature.

Suitable infrared detectors 12 are known in the art, such as infrared cameras and the like. Similarly, it is well known to provide a variety of incubators 18 and controlled temperature chambers with coolers or heaters 20 for holding microorganisms, cultured cells, etc. at a desired temperature. The data processor 14 may be any suitable device capable of receiving data from the infrared detector 12 and processing those data as will be described below, including personal computers, mainframe computers, and the like.

### Example 2

The system described in Example 1 was used to determine the thermal properties of bacterial growth media in the presence and absence of bacteria. For that purpose, a series of inocula of Escherichia coli were established in Mueller-Hinton broth (MHB) in ten-fold dilutions, ranging from 10² to 10⁸ colony-forming units (CFU)/ml. Control wells contained MHB alone. Temperature measurements obtained using the system of Example 1 were normalized to reduce dependence on location, acquisition time, initial temperature, and surrounding temperature; and correlated with the normalized acquisition time (t). The slope of the correlation was found to be a function of the normalized released-thermal-energy by the media, referred to as the Energy Number (Eₙ), which was found to be directly related, to the interaction of the temperature with the surrounding and the measurement time, set forth in the following relation:

Energy Number∝f(T)·g(t)

That temperature function is a non-dimensional number that compares temperature changes in each well on the same relative scale, defined as:

$$Δ T = 1 - \frac{T - {T}_{a}}{{T}_{i} - {T}_{a}}$$

where ΔT is the relative temperature change. Similarly, the time scale was normalized with respect to a time constant (τ) derived from the slope of temperature cooling time rate for the media without bacteria. The normalized time was expressed in the logarithmic function:

$${t}^{*} = 1 - {e}^{\frac{- t}{τ}}$$

where t* is the normalized time. Then:

f(T)=Slope×g(t)

f(T)=1−ΔT,Slope=(1−Eₙ),g(t)=t*

The Energy Number Eₙ was thus defined as:

$${E}_{n} = \frac{{E}_{M}}{h {A}_{s} ( {T}_{i} - {T}_{a} )}$$

where E_{M} is the metabolism energy introduced to the media by the bacterial activity', h is the convection heat transfer coefficient, and A_{S} is the surface area of the well. The metabolism energy E_{M} was defined as:

E_{M}=E′_{M}F(N)

where E′_{M} is the energy produced by one microorganism cell, N is the bacterial concentration, and F(N) is a function of the number of bacteria. The value Eₙ defined below can be measured:

$${E}_{n} = \frac{Δ f ( T )}{Δ g ( t )}$$

where Δf(T) is the change in normalized temperature and Δg(t) is the change in normalized time.

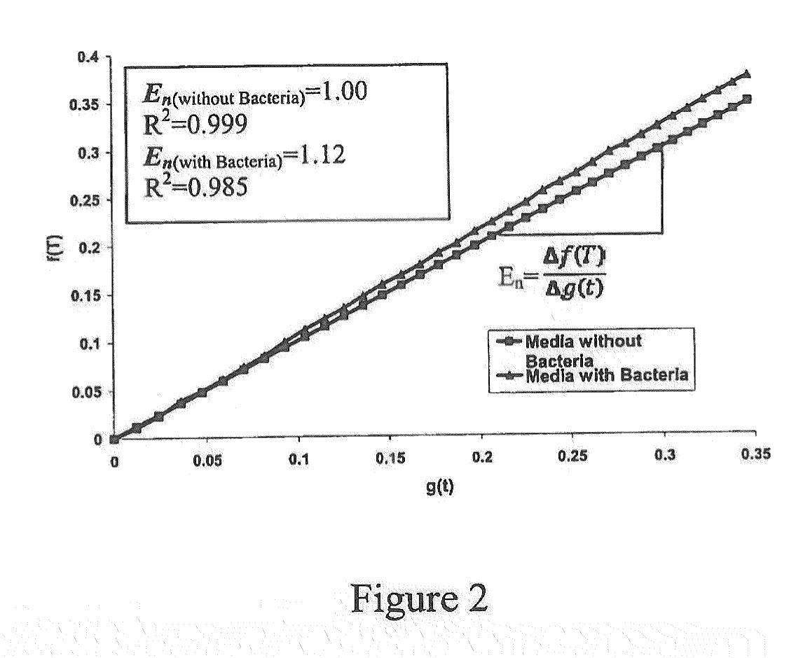

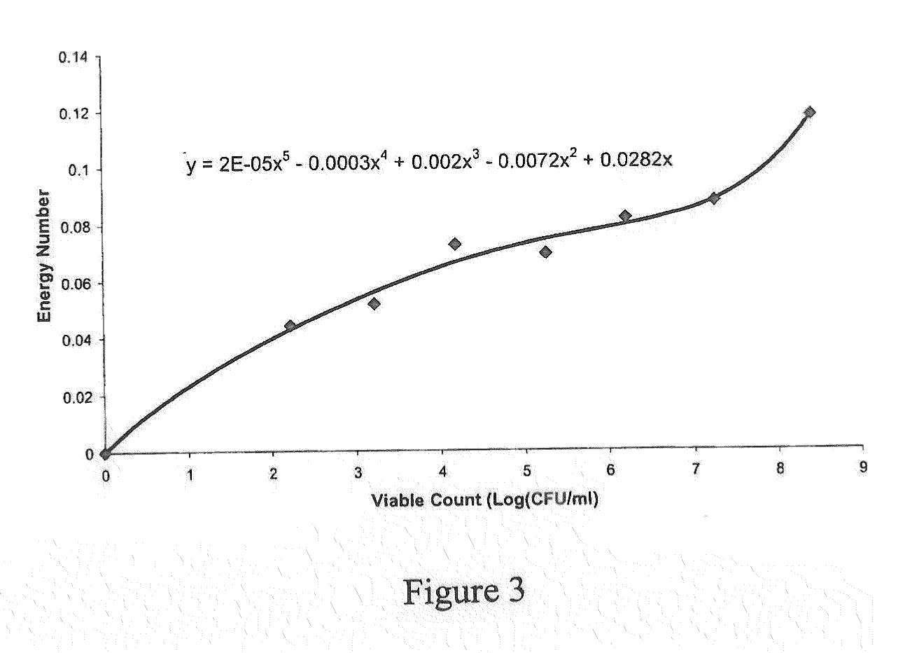

A representative experiment is shown in FIG. 2, showing a plot of normalized temperature [f(T)] versus normalized time [g(t)]. The results demonstrated that the Energy Number (Eₙ) was significantly increased for media containing bacteria in comparison to media lacking bacteria (P<0.0001, Student's t test). Still further, it was found that Energy Number (Eₙ) was highly coixelated to the viable count when the bacteria were in the lag phase of growth, with Eₙ ranging from 0-0.1 (see FIG. 3).

### Example 3

A 24-well culture dish was prepared by painting with a smooth satin black, to reduce reflection, and inoculated with E. coli at increasing concentrations as set forth in Table 1. Culture conditions were as described in Example 2. Only the center 8 wells were inoculated. Thermal imaging was recorded by an Avio Photonic Detector, TVS-8500. Images were taken at 1 minute intervals for a total of 30 minutes, to ascertain the effect of time.

TABLE 1
Concentration of E. coli per well

| Well # | Concentration (cfu/ml) |
|---|---|
| 1, 2 | 1.67 × 10² |
| 3, 4 | 1.68 × 10⁴ |
| 5, 6 | 1.92 × 10⁶ |
| 7, 8 | 2.40 × 10⁸ |

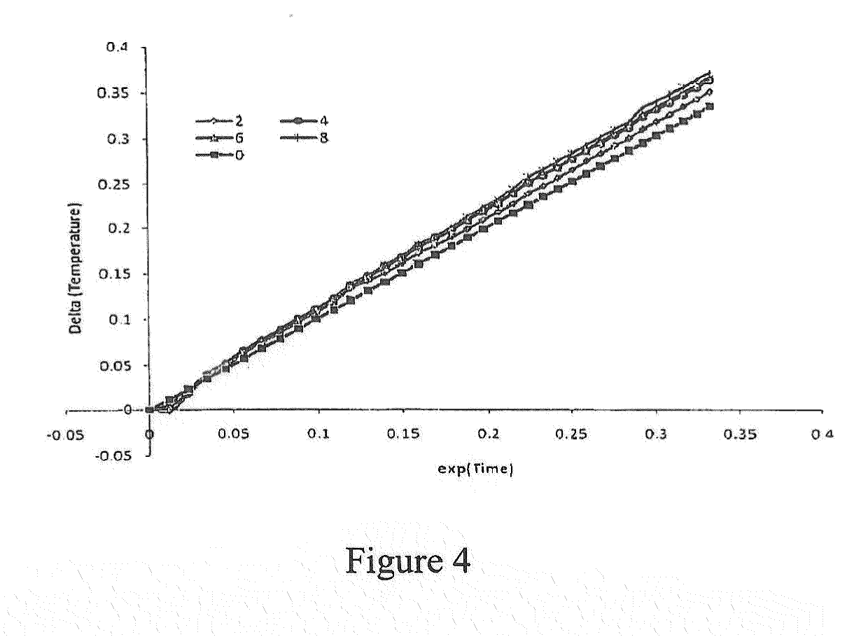

As shown in FIG. 4, the concentration of bacteria could be differentiated according to temperature evolution over time. A linear relationship was observed between the normalized temperature and the normalized time. The method was further demonstrated to differentiate between media without bacteria and media with increasing concentrations of bacteria.

### Example 4

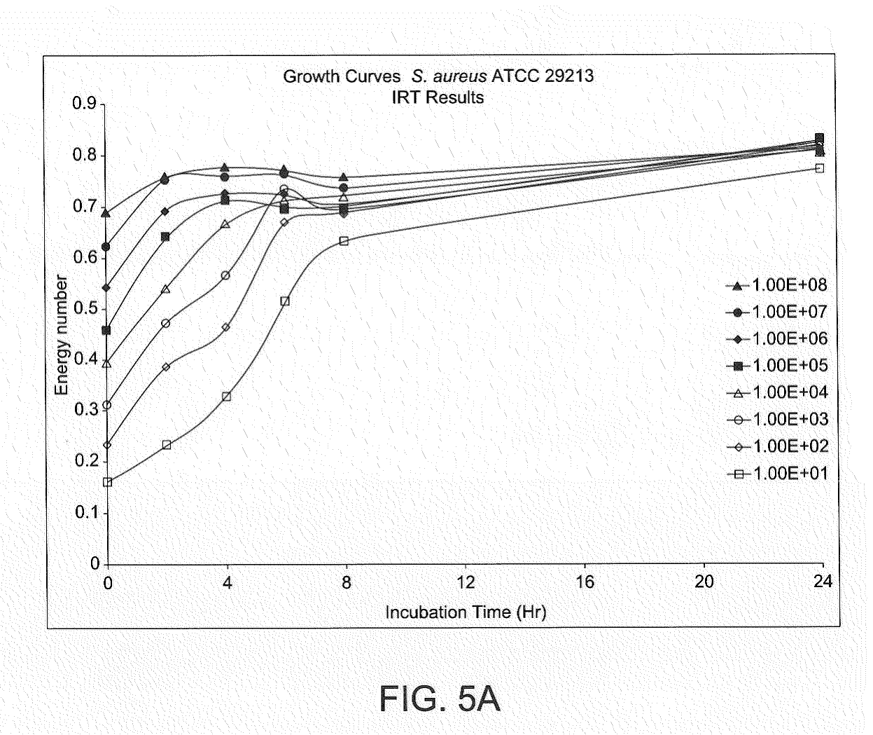

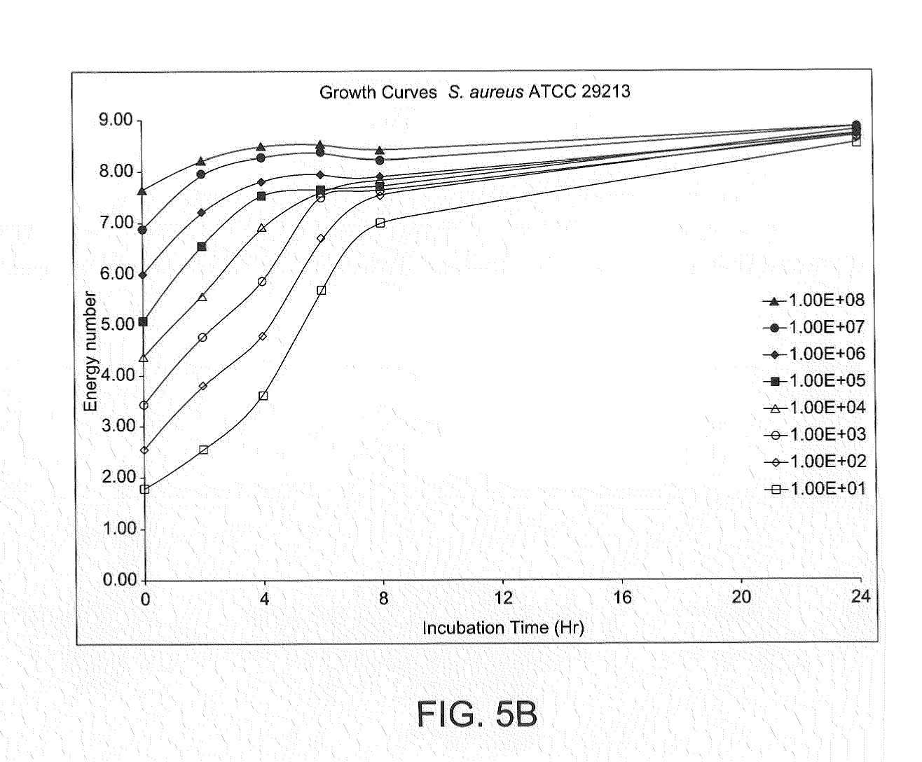

The presently described system 10 was used to determine the thermal properties of the growth media in the absence and presence of a representative grain positive bacterium, Staphylococcus aureus (S. aureus). Inocula ranging from 10¹ to 10⁸ CFU/ml in ten-fold dilutions were prepared in MHB. The same experimental method that was used for Example 2 was applied to measure thermal properties of Staphylococcus aureus. Energy number (Eₙ), ranging from 0.15 to 0.825, shown in FIG. 5a was highly correlated with the bacterial viable count shown in FIG. 5b.

### Example 5

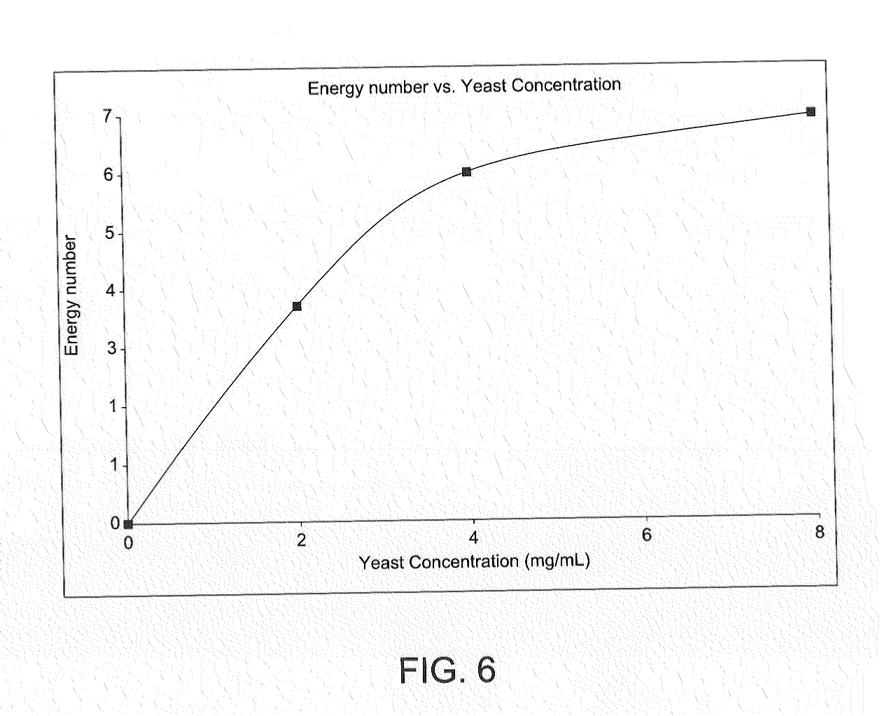

The described system 10 was used to determine the thermal properties of the growth media in the absence and presence of a commercially available active dry baking yeast. 24 well plates series of inocula of yeast were prepared in (NM) with three different concentrations (2, 4, and 8 mg/ml) and one control. FIG. 6 shows that the Eₙ value between 3.7 and 6.9 was highly correlated with the yeast concentration.

### Example 6

The present system 10 is used to measure the growth of a yeast, Candida albicans, in RPMI 1640 with L-glutamine broth without bicarbonate, a medium recommended for antifungal susceptibility testing. Thermal signatures of modified RPMI 1640 broth alone and RPMI 1640 with different inocula of the reference strain C. albicans ATCC 90028 in ten-fold dilutions ranging from 10² to 10⁸ CFU/ml are determined. Growth of the yeast in 96-well microtiter plates at 35° C. is monitored every 2 h for 24 h. Correlations between energy released from the surface of the media and the yeast cell counts are determined. In this fashion, system 10 is used for monitoring death of the yeast cells in real time during exposure to a representative antifungal agent (e.g. amphotericin B, flucytosine, ketoconazole, or fluconazole). The data are used to develop a mathematical model to describe the thermodynamics of yeast cell growth and death.

### Example 7

The present system 10 is used to measure the growth of the marine dinoflagellate, Alexandrium fundyense, which is a eukaryotic algae responsible for red tide. Cultures are grown in 172 medium made with 0.2 μm filtered seawater (31 practical salinity units) and modified by the addition of H₂SeO₃ and CuSO₄, both to final concentrations of 10⁻⁸M. Cultures are incubated at 20° C. on a 14 h light:10 h dark cycle. The cultures are removed from the incubator at different time intervals and allowed to equilibrate at an ambient room temperature (25° C.). During this 3 minutes transient heating period, IR images are taken using an infrared detector. Thermal properties of the media in the absence and presence of 10-fold dilutions of A. fundyense are determined. The proliferation of the dinofiagellate over several days under the above culture conditions is evaluated and the correlation between energy released from the surface of the media and microscopic cell counts is determined.

### Example 8

The present system 10 is used to measure the growth of the marine dinoflagellate, Alexandrium fundyense, which is a eukaryotic algae responsible for red tide. Cultures are grown in f/2 medium made with 0.2 μm filtered seawater (31 practical salinity units) and modified by the addition of H₂SeO₃ and CuSO₄, both to final concentrations of 10⁻⁸ M. Cultures are incubated at 20° C. on a 14 h light:10 h dark cycle. The cultures are removed from the incubator at different time intervals and allowed to equilibrate at an ambient cold room temperature (4° C.). During this 3 minutes transient cooling period, IR images are taken using an infrared detector. Thermal properties of the media in the absence and presence of 10-fold dilutions of A. fundyense are determined. The proliferation of the dinoflagellate over several days under the above culture conditions is evaluated and the correlation between energy released from the surface of the media and microscopic cell counts determined.

### Example 9

The present system 10 is used to measure the growth of the human monocyte cell line THP-1 (ATCC TIB-202) and the human colorectal adenocarcinoma cell line HT-29 (ATCC HTB-38). Cell cultures of THP-1 are grown at 37° C. in RPMI 1640 media supplemented with 2-mercaptoethanol (0.05 mM) and fetal bovine serum (10%). Cell cultures of HT-29 are grown at 37° C. in modified McCoy's 5a media supplemented with fetal bovine serum (10%). Thermal properties of the respective media in the absence and presence of each cell line in concentrations ranging from 5×10⁴ to 1×10⁶ viable cells/ml are determined using System 10. The replication of both cell lines is monitored daily over two to three days in a 96-well microtiter plate under the above culture conditions. The correlation between energy released from the media and microscopic cell counts is determined.

### Example 10

It was desired to evaluate utility of the system in a real-time method to measure bacterial growth and death as a function of exposure to an antimicrobial. For that purpose, time-kill studies were performed using E. coli cultures, established substantially as set forth in Example 2. A series of microtiter plates were inoculated with an overnight culture of a representative gram-negative E. coli strain to achieve a starting inoculum of approximately 2×10⁶ CFU/ml per well. Test wells were exposed to nine concentrations of an antimicrobial agent of a representative fluoroquinolone (ciprofloxacin) ranging from 0- to 32-fold minimum inhibitory concentration (MIC). Five of those treatments (0, 5, 1, 2, 4, and 8-fold MIC) were subcultured for determination of viable cell counts. Control wells received no ciprofloxacin. The plates were incubated at 37° C. for a predetermined time interval (0, 2, 4, 6, 8, and 24 hours), removed from the incubator, allowed to equilibrate to an ambient cold room temperature (4° C.), and imaged over a 3 minute time period using an infrared detector (FLIR Infrared Detector, Model No. SC4000, N. Billerica, Mass.). Post-imaging, the contents of the designated wells were removed and the number of viable bacteria in each well was determined using drop and filter count methods.

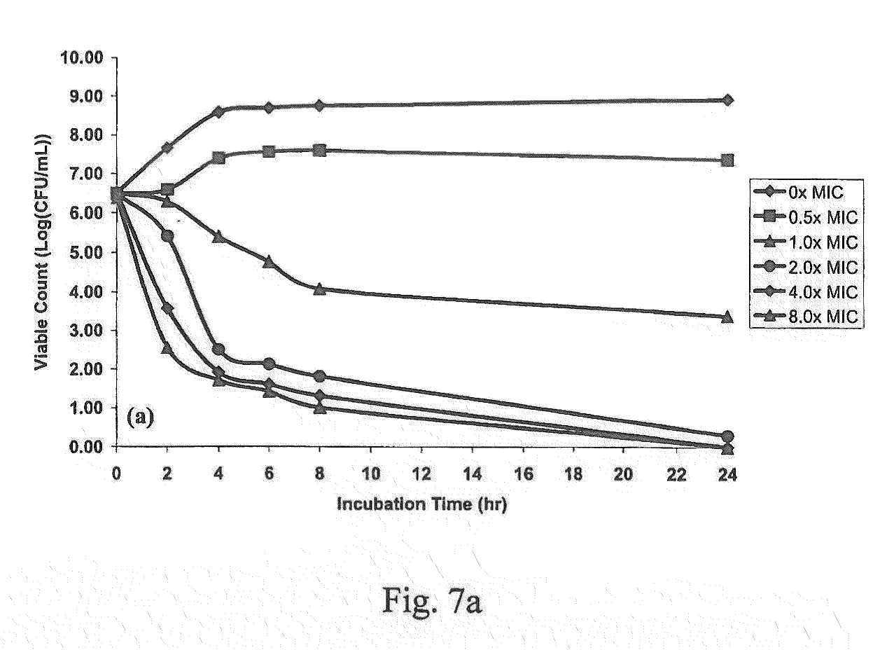

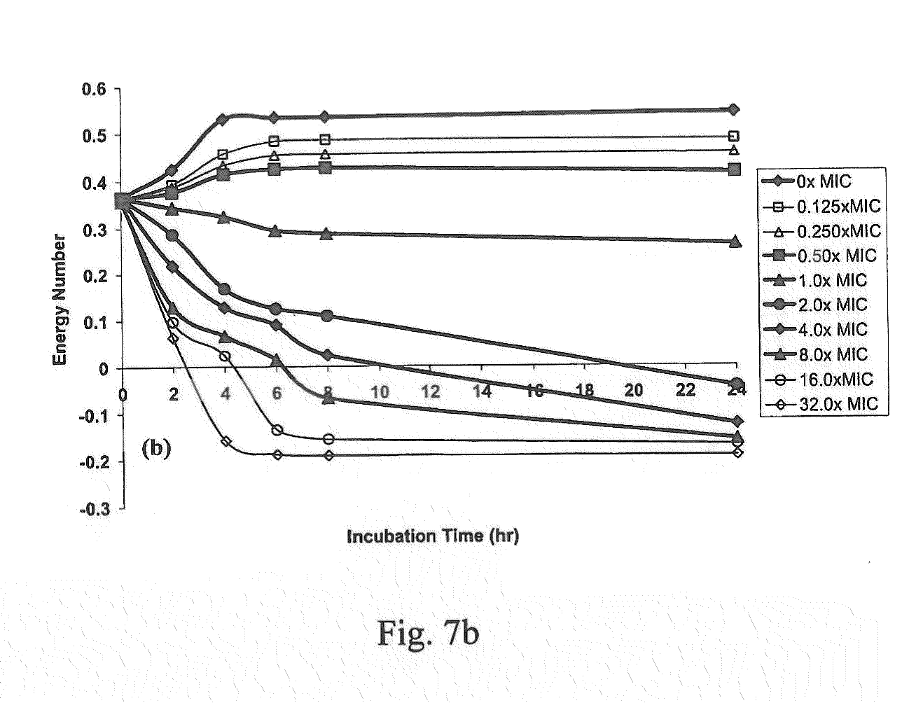

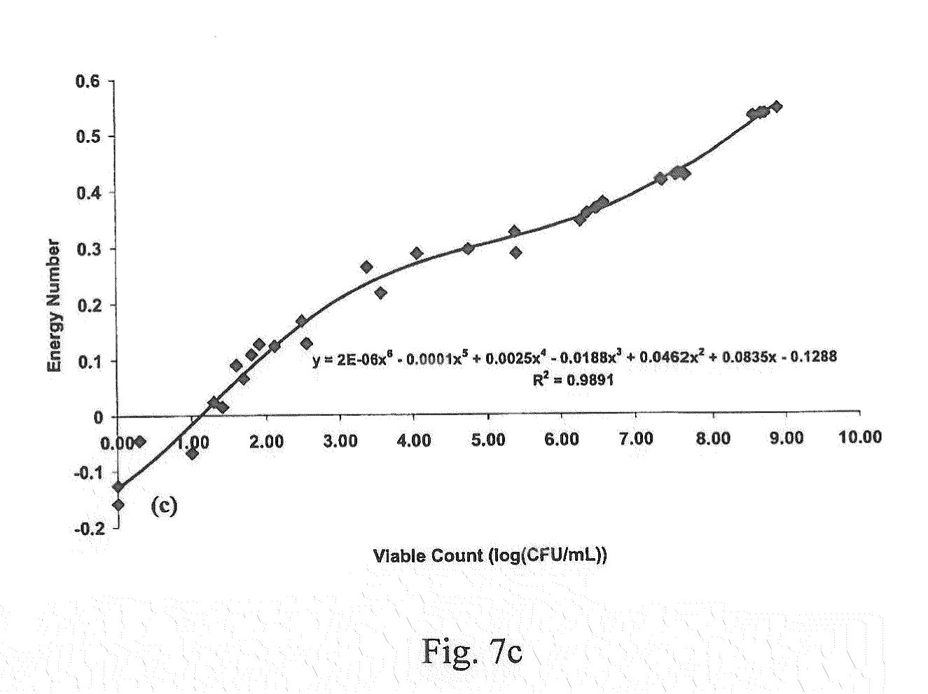

FIGS. 7a and 7b set forth the changes in bacterial viable counts and the Energy Number of the medium over time, respectively. The Energy Number of the medium over time as determined from thermodynamic modeling of the IR images collected by the detector was highly correlated (R²=0.9891) to the viable counts obtained following 24 hr incubation (see FIG. 7c). The IRT results demonstrated net bacterial growth at the lowest (0.125 and 0.25-fold MIC) concentration, and rapid bacterial killing at the highest (16.0 and 32.0-fold MIC) concentration.

### Example 11

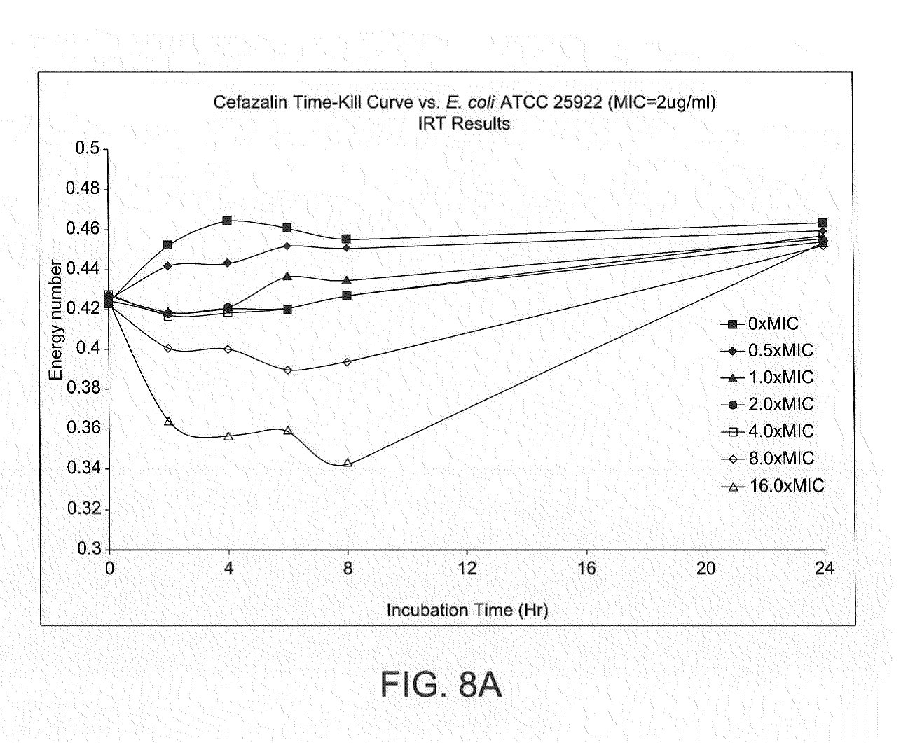

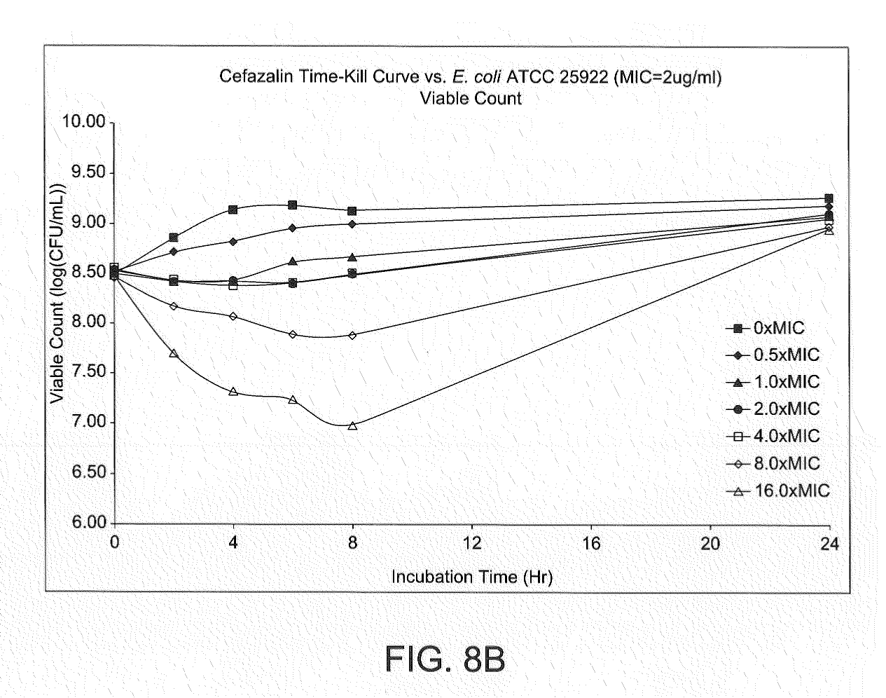

A series of microtiter plates were inoculated with an overnight culture of a representative gram-negative E. coli strain to achieve a starting inoculum of approximately 2×10⁸ CFU/ml per well. These test wells were exposed to nine different concentrations of an antimicrobial agent of a representative cephalosporin (cefazolin) ranging from O— to 16-fold the minimum inhibitory concentration (MIC). Wells containing five of those concentrations (0.5, 1, 2, 4, and 8-fold MIC) were sub-cultured for determination of viable cell counts. Control wells contained no cefazolin. All plates were incubated at 37° C. and removed from the incubator at six different time intervals (0, 2, 4, 6, 8, and 24 hours) and allowed to equilibrate at an ambient cold room temperature (4° C.). During this 3 minutes transient cooling period, IR images were taken using an infrared detector (FUR Infrared Detector, Model No. SC4000, N. Billerica, Mass.). Our IRT results (FIG. 8a) were compared favorably with viable counts determined by the drop count method (FIG. 8b).

### Example 12

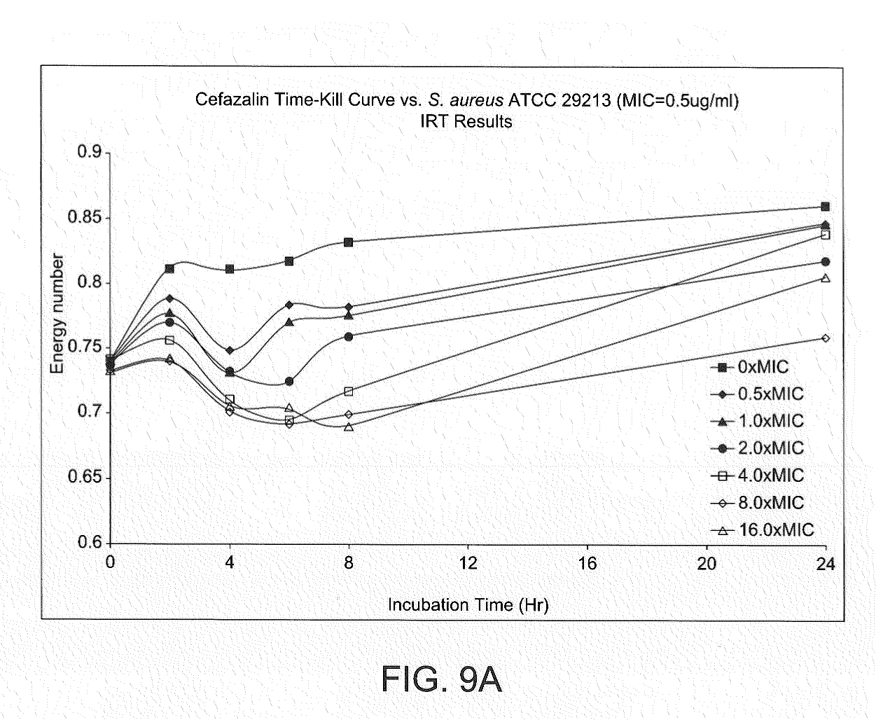

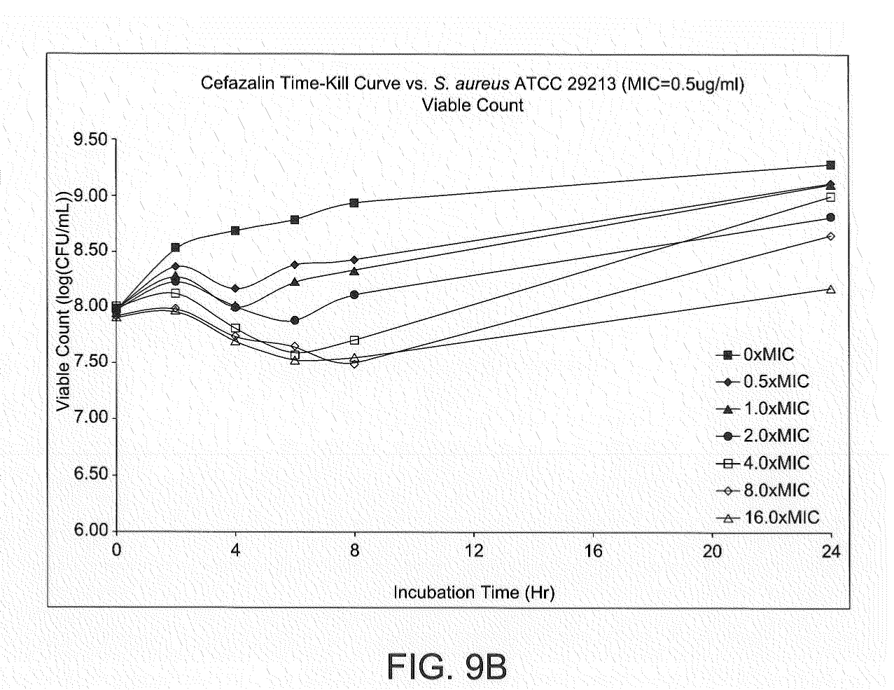

A series of microtiter plates were inoculated with an overnight culture of a representative gram-positive S. aureus steam to achieve a starting inoculum of approximately 2×10⁸ CFU/ml per well. These test wells were exposed to nine different concentrations of an antimicrobial agent of a representative cephalosporin (cefazolin) ranging from O— to 16-fold the minimum inhibitory concentration (MIC). Wells containing five of those concentrations (0.5, 1, 2, 4, and 8-fold MIC) were sub-cultured for determination of viable cell counts. Control wells contained no cefazolin. All plates were incubated at 37° C. and removed from the incubator at six different tune intervals (0, 2, 4, 6, 8, and 24 hours) and, allowed to equilibrate at an ambient cold room temperature (4° C.). During this 3 minutes transient cooling period, IR images were taken using an infrared detector (FLIR Infrared Detector, Model No. SC4000, N. Billerica, Mass.). Our IRT results (FIG. 9a) were compared favorably with viable counts determined by the drop count method (FIG. 9b).

There is accordingly provided a method for detecting and quantitating a viable microorganism in a fluid sample. The method advantageously is non-contact, and does not require any chemical additions or destruction of a sample to provide information regarding viable microorganism numbers. Still further, the method provides real-time detection and quantitation of viable microorganisms in a sample, rather than requiring lengthy incubation periods as are necessary in conventional colony count methods. The method finds use in a variety of applications, including evaluation of test agents for microbiocidal, microbiostatic, or growth enhancer properties.

One of ordinary skill in the art will recognize that additional embodiments of the mention are also possible without departing from the teachings herein. This detailed description, and particularly the specific details of the exemplary embodiments, is given primarily for clarity of understanding, and no unnecessary limitations are to be imported, for modifications will become obvious to those skilled in the art upon reading this disclosure and may be made without departing from the spirit or scope of the invention. Relatively apparent modifications, of course, include combining the various features of one or more figures or examples with the features of one or more of other figures or examples.

1. A method for quantifying an amount of a viable microorganism, comprising:
subjecting a fluid sample suspected of containing a viable microorganism to a temperature change; and
correlating the temperature change in the fluid sample to the amount of the viable microorganism contained in the fluid sample.

2. The method of claim 1, comprising the steps of:
bringing the fluid sample to a first temperature;
transferring the fluid sample to a second temperature that is different than the first temperature;
after said step of transferring, measuring a temperature change in the fluid sample over a predetermined period of time; and
correlating the temperature change in the fluid sample to the amount of the viable microorganism contained in the fluid sample.

3. The method of claim 2, wherein the fluid sample is held at the first temperature for a sufficient period of time to place the fluid sample in a state of thermal equilibrium.

4. The method of claim 3, wherein the first temperature is an optimum growth temperature of the microorganism studied.

5. The method of claim 4, wherein the temperature is from about 35° C. to about 37° C.

6. The method of claim 2, wherein the second temperature is sufficient to induce a thermal transient state in the microorganism.

7. The method of claim 6, wherein the second temperature is a controlled ambient temperature.

8. The method of claim 7, wherein the second temperature is from about 4° C. to about 25° C.

9. The method of claim 2, wherein the temperature change is measured by the steps of:
holding the fluid sample at the second temperature for a predetermined time period; and
measuring a temperature change in the fluid sample at spaced time intervals during the predetermined time period.

10. The method of claim 9, wherein the temperature change in the fluid sample is measured during the step of holding the fluid sample at the second temperature for the predetermined time period.

11. The method of claim 10, wherein the step of measuring the temperature change is accomplished by acquiring a plurality of sequential thermal images of the fluid sample at the spaced time intervals.

12. The method of claim 11, wherein the plurality of sequential thermal images are acquired by infrared thermography.

13. The method of claim 2, wherein the step of correlating comprises relating a plotted slope of the normalized temperature change against the normalized predetermined time period to an amount of thermal energy released from the fluid sample.

14. The method of claim 13, wherein the amount of thermal energy released from the fluid sample is correlated to the plotted slope of a normalized temperature change of the sample against a normalized predetermined time period according to the formula:
$${E}_{n} = \frac{Δ f ( T )}{Δ g ( t )}$$
where Δf(t) is change in normalized temperature and Δg(t) is change in normalized time.

15. A method for determining the effect of a test substance on growth or viability of a microorganism, comprising:
suspending a predetermined amount of a viable microorganism in a fluid sample;
adding a predetermined amount of a test substance to the fluid sample;
subjecting the fluid sample to a temperature change; and
correlating the temperature change in the fluid sample to an amount of viable microorganism contained in the test fluid to determine the effect of the test substance on the amount of the viable microorganism contained in the fluid sample.

16. The method of claim 15, comprising the steps of:
bringing the fluid sample to a first temperature;
transferring the fluid sample to a second temperature that is different than the first temperature;
after said step of transferring, measuring a temperature change in the fluid sample; and
correlating the temperature change in the fluid sample to an amount of viable microorganism contained in the fluid sample to determine the effect of the test substance on the amount of the viable microorganism contained in the fluid sample.

17. The method of claim 15, wherein the test substance is suspected of being a microbicidal composition or compound, a microbiostatic composition or compound, or a microbial growth enhancer.

18. The method of claim 16, wherein the first temperature is maintained for a sufficient period of time to place the fluid sample in a state of thermal equilibrium.

19. The method of claim 18, wherein the first temperature is an optimum growth temperature of the microorganism studied.

20. The method of claim 19, wherein the first temperature is from about 35° C. to about 37° C.

21. The method of claim 16, wherein the second temperature is sufficient to induce a thermal transient state in the microorganism.

22. The method of claim 21, wherein the second temperature is a controlled ambient temperature.

23. The method of claim 22, wherein the second temperature is from about 4° to about 25° C.

24. The method of claim 16, wherein the temperature change is measured by the steps of:
holding the fluid sample at the second temperature for a predetermined time period; and
measuring a temperature change in the fluid sample at spaced time intervals during the predetermined time period.

25. The method of claim 24, wherein the temperature change in the fluid sample is measured during the step of holding the fluid sample at the second temperature for the predetermined time period.

26. The method of claim 24, wherein the step of measuring a temperature change is accomplished by acquiring a plurality of sequential thermal images of the fluid sample at the spaced time intervals.

27. The method of claim 26, wherein the plurality of sequential thermal images are acquired by infrared thermography.

28. The method of claim 16, wherein the step of correlating comprises relating a plotted slope of a normalized temperature change against a normalized predetermined time period to an amount of thermal energy released from the fluid sample.

29. The method of claim 28, wherein the amount of thermal energy released from the fluid sample is correlated to the plotted slope of normalized temperature change against normalized predetermined time period according to the formula:
$${E}_{n} = \frac{Δ f ( T )}{Δ g ( t )}$$
where Δf(T) is change in normalized temperature and Δg(t) is change in normalized time.
